(19) United States
(12) Patent Application Publication
(10) Pub. No.: US 2018/0078602 A1
(43) Pub. Date: Mar. 22, 2018

(54) METHODS AND COMPOSITIONS FOR EXTENDING THE EFFICACY OF PHOSPHODIESTERASE INHIBITORS FOR TREATING ERECTILE DYSFUNCTION, AND COMPOSITIONS FOR INHIBITING THE ONSET AND SLOWING THE PROGRESSION OF ERECTILE DYSFUNCTION

(71) Applicant: K.L.R.M., LLC, Long Beach, CA (US)

(72) Inventors: Mauro Milchteim, Chapel Hill, NC (US); Floyd Alexander Katske, Los Angeles, CA (US); Jacob Rajfer, San Pedro, CA (US)

(21) Appl. No.: 15/554,161

(22) PCT Filed: Mar. 4, 2016
(86) PCT No.: PCT/US16/20983

§ 371 Date: Aug. 28, 2017

Related U.S. Application Data
(60) Provisional application No. 62/128,269, filed on Mar. 4, 2015.

(51) Int. Cl. A61K 36/9068; A61K 31/198; A61K 36/185; A61K 36/77; A61K 45/06

(52) U.S. Cl. CPC A61K 36/9068; A61K 31/198; A61K 45/06; A61K 36/77; A61K 36/185

(57) ABSTRACT

The duration of treatment of erectile dysfunction (“ED”) with phosphodiesterase inhibitors can be extended by concomitant administrations of compositions comprising ginger, and a compound that produces L-arginine in vivo. Preferred compositions of the present invention comprise ginger, L-citrulline, Muira Puama and Paullinia cupana. The compositions are administered in a pharmaceutically effective amount for a sufficient period of time to treat or as a prophylaxis against ED or conditions associated with tissue fibrosis.

This application relates to co-pending U.S. patent application Ser. No. 13/786,117, filed Mar. 5, 2013, which is a Continuation-In-Part of International Application Serial No. PCT/US2011/056965, filed Oct. 16, 2011 (International Publication No. WO 2012/067745 A1, published May 24, 2012, or “Rajfer et al”), which claims priority of U.S. patent application Ser. No. 12/907,113, filed Oct. 19, 2010. International Publication No. WO 2012/067745 A1 is incorporated by reference as if reproduced in full herein below. New formulations are disclosed therein comprising ginger with L-citrulline and/or L-arginine that are useful for treating erectile dysfunction. Additional ingredients are present in certain of the aforementioned compositions. Research discussed herein has further elucidated the beneficial effects of the earlier inventions set forth in the Rajfer et al publication, and new and surprising discoveries are reflected in new inventions described herein. In particular, further research has been conducted to elucidate the histological effects of certain natural products, and to seek out new and/or better formulations and treatment methodologies to enable treatment of ED, ARED, and certain fibrotic diseases such as arteriosclerosis. This led to the surprising discovery disclosed herein that those compositions and the new compositions disclosed herein can be used to extend the duration of efficacy of PDE-I compounds in treating ED and/or can extend the duration of use of PDE-I compounds.

### FIELD OF THE INVENTION

The present inventions relate to compositions and methods for extending the duration of efficacy of phosphodiesterase inhibitor (“PDE-I”) compounds in treating erectile dysfunction (“ED”), and includes compositions for and methods for inhibiting the onset, and/or slowing the rate of development of ED as well as age related erectile dysfunction (“ARED”). In particular, the invention relates to compositions comprising ginger (also referred to as “G”), and L-citrulline (L-citrulline can be replaced partially or completely with L-arginine; L-citrulline and/or L-arginine is also referred to herein as “C”). Preferred compositions also include Muira Puama (“M”) and Paullinia cupana (“P”). Compositions comprising GC and GCMP can be used to retard, halt and/or reverse histological and functional characteristics of ED, ARED and certain fibrotic diseases. GCMP compositions may be combined with or administered concomitantly with PDE-I compound, such as a type 5 phosphodiesterase (“PDE5”) inhibitor compound, to extend the duration of efficacy and/or duration of treatment with PDE-I compounds.

### BACKGROUND

Referring to the Rajfer et al Publication (Intl Pub. No. WO 2012/067745 A1), it is noted that impotence or erectile dysfunction (ED) is a problem that most men will face at some time in their life. Erectile dysfunction (ED) associated with the aging process is characterized histologically within the cavernosa by a progressive apoptosis or loss of the corporal smooth muscle cells (SMC) and replacement of these cells with collagen (this may be referred to as fibrosis). It has been estimated that once approximately 15-20% of the corporal SMCs have been lost, venous leakage or corporal veno-occlusive dysfunction (CVOD) becomes clinically apparent. It has been demonstrated that as these aging-related histological changes begin to occur in the cavernosa, the SMCs themselves attempt to combat these apoptotic and fibrotic changes by upregulating inducible nitric oxide synthase (iNOS). It is believed that the high output of nitric oxide (NO) produced intracellularly by iNOS can act in this setting and in other conditions as an anti-fibrotic factor. This anti-fibrotic effect of iNOS is evident not only in aging related ED (ARED) but also in the bilateral cavernosal resection rat model of ED where the histological changes that occur in the cavernosa resembles a more accelerated version of ARED. ARED may also be referred to as age-related ED.

The major reason ED manifests itself, regardless of the age of onset, is due to an alteration in the corporal cavernosal smooth muscle (CSM), which is located within the cavernosal or corporal bodies of the penis.

The function of the CSM in the erectile process is to receive and trap blood entering the corporal bodies. When the CSM begins to degrade it cannot maintain its relaxation long enough to provide sufficient tumescence. The first recognition that one's erectile mechanism is worsening is the increase in time that it takes for one to achieve subsequent erections. This time in between subsequent erections is called the refractory period and it is the first indication that the CSM tissue is changing for the worse.

When the CSM cells begin to undergo oxidation and deteriorate either as a result of aging or some other cause, the CSM begin to induce an enzyme called inducible nitric oxide synthase (iNOS), which produces nitric oxide in high quantities within the cells that begins to combat the oxidative stress. This induction of nitric oxide (NO) by iNOS is different from the NO that is found in the nerves of the body including the nerves that innervate the CSM cells. NO in the nerves is produced by a related enzyme called neuronal nitric oxide synthase (“nNOS”) and it only releases NO when the patient is sexually stimulated. This NO from nNOS is the major chemical that is involved in the relaxation of the CSM cells and hence is required for the initiation and maintenance of a normal erection. Therefore, while nNOS is normally present in the nerves innervating the penis, iNOS is normally not present in the CSM cells of the penis and is only induced by the CSM cells themselves when the cells experience oxidative stress. However, when iNOS is induced as seen in U.S. Pat. No. 5,594,032, human erectile dysfunction can be ameliorated by treatment with iNOS, inducers of iNOS or iNOS cDNA. Further background on sexual dysfunction, urogenital disease, and ED and related treatments can be found in U.S. Pat. No. 6,133,281, U.S. Pat. No. 6,007,824, U.S. Patent Publication 2005/0085486, and Schwartz, Eric, et al., “Sildenafil Preserves Intracorporeal Smooth Muscle After Radical Retropubic Prostatectomy,” The Journal of Urology, Vol 171, pp. 771-774, February 2004.

With reference to U.S. Patent Publication 2005/0085486, fibrotic disease is linked to reproductive disorders and cardiovascular disease, which are both prevalent in aging males. The ubiquitous and long felt need to treat sexual dysfunction has led to surgical and pharmacological treatment approaches. The ongoing commercial success of prescription medications under the trademarks VIAGRA® (sildenafil), LEVITRA® (vadenafil) and CIALIS® (tadafil) for treatment of ED demonstrates the long felt and widespread need for effective treatments for ED, particularly for patients that present with ED symptoms advanced sufficiently that erections of satisfactory duration at the desired time can only be reliably accomplished by taking the prescription drug.

Thus, there remains a ubiquitous and long-felt need to treat ED before it progresses to the point where pharmacological and/or other medical intervention is required in order to have desired sexual performance. Nevertheless, current ED drugs, such as VIAGRA® and CIALIS®, are generally prescribed only after the patient has presented with symptoms of ED. These drugs belong to a class of drugs called Type 5 phosphodiesterase (PDE5) inhibitors. PDE5 is an enzyme that breaks down cGMP once it is formed. PDE5 inhibitors (e.g., Viagra) prevent the cGMP from breaking down, so the effect of the cGMP on the tissues is enhanced. With regards to erectile function, cGMP is formed within the CSM from a reaction that is initiated by the NO that is released from the cavernosal nerve following sexual stimulation. As noted above, the NO that begins the erectile response comes from the enzyme nNOS that is located in the nerve endings. NO goes into the CSM cells and causes a reaction to occur in these CSM cells. NO activates the enzyme soluble guanylyl cyclase (sGC) in the cytoplasm of the CSM and this enzyme in turn converts guanosine triphosphate (GTP) into cyclic guanosine monophosphate (cGMP). An increase in cGMP stimulates protein kinase G to phosphorylate potassium and calcium channels causing a decrease in cytosolic calcium, dilation of the helicine arterioles, and the relaxation of the trebecular smooth muscle where all the CSM cells are located. As noted above, the relaxation of the smooth muscle leads to an increase in the intracavernosal volume, initiating the erectile process. Normally, endogenous PDE5 enzyme degrades cGMP which reverses the relaxation of the smooth muscle cells and leads to loss of erection whereas the ingestion of PDE5 inhibitors (“PDE5-I”) prior to sexual stimulation prevent the degradation of the cGMP that is formed thereby, thus prolonging any CSM relaxation and enhancing any erectile response.

Currently, one suffering from ED needs to see a doctor and get a prescription for an ED treatment in advance in order to be prepared for a satisfying experience. Onset and duration of the effects of PDE5-I depend on the specificity of the compound. While use of a PDE5-I compounds is considered the “first line treatment of ED,” there are notable side effects (headache, flushing, dyspepsia, rhinitis, visual disturbances, back pain, etc.) and adverse interactions that can limit or bar their use (e.g., patients taking nitrates with a PDE5-I can experience hypotension and syncope. See Dorsey, Philip et al., “Phosphodiesterase type 5 (PDE5) inhibitors for the treatment of erectile dysfunction,” Expert Opinion, Pharmacother. (2010), 11(7): pp 1109-1122). It has been noted that a substantial number of patients for which PDE-I compounds are initially efficacious find over time that their ED symptoms worsen so that the PDE-I compounds become less effective or not effective enough to yield satisfaction. This leads to patients stopping or reducing use of PDE-I compounds. Since PDE-I compound use over a prolonged period of time has some antifibrotic benefits, patient noncompliance deprives patients of such benefits. Since most men will at some time in their life get ED mainly as the result of CSM deterioration secondary to the aging process, it must be determined whether or not it is desirable to treat men who are asymptomatic but whose refractory period has begun to increase—a subtle sign that the CSM is begin to undergo deterioration in order to slow or prevent the progression of this deterioration and the forthcoming ED. For patients who already have noticeable ED symptoms, it may be desirable to slow if not stop or reverse the progression of the ED.

CONFLICTS IN THE PRIOR ART. There are numerous conflicting reports regarding the alleged ability of certain natural products, alone or in combination, to enhance sexual performance and/or temporarily reverse ED symptoms. Multiple reports of enhanced sexual virility from ingestion of natural products have been found to be unsubstantiated; in some cases, alleged beneficial compositions have been found to have the opposite or even toxic effects. For example, reports about alleged sexual health benefits of mixtures of ginseng, gingko balboa, and/or other natural products, including ginger and/or amino acids, have been contradicted by modern scientific research, making the use of herbs and herb mixtures unpredictable and associated claims unreliable. Rigorous and systematic scientific methodology, such as that required to gain acceptance of medical professionals, has been lacking. Medical professionals must be comfortable with the reliability of claimed benefits of natural products in order to recommend them to patients. Such reliability comes from provision of a rational scientific basis for claims, properly controlled human studies and/or animal studies (including histological analysis when possible). Nevertheless, some natural products have been found in scientific research to have useful bioactivity, and research is ongoing. For example, the U.S. National Institutes of Health has ongoing programs to fund research into determining the true bioactivity of many natural products, while also assuring the integrity of the scientific research and analysis of data. Further, certain animal models of ED have been demonstrated as proper surrogates for human ED and/or fibrotic diseases. Studies utilizing such animal models, particularly when conducted in parallel with PDE5-I studies and/or using other scientifically validated methodologies, are particularly valuable for confirming usefulness in humans.

With respect to ginger, also known as Zingiber officinale roscoe (ZOR), numerous folk remedies include it with a plethora of other ingredients and combinations for treating sexual dysfunction. For example, formulations of ginger with Danzhixiaoyao Wan taught for treating sexual dysfunction are contradicted by scientific studies that show that the latter actually inhibits nitric oxide production, i.e., could cause erectile dysfunction (see Liao, Hui et al., “Elucidation of Danzhixiaoyao Wan and its Constituent Herbs on Antioxidant Activity and Inhibition of Nitric Oxide Production,” eCam, Advanced Access publication Jan. 9, 2007). Nevertheless, the same article indicates that ZOR (ginger) at 500 mg/L “seemed to stimulate nitric oxide production” but had no effect at 50 mg/L. Further, it has been reported that high doses of ginger combined with the iNOS stimulant lipopolysaccharide (LPS) could increase nitric oxide production, yet this conflicted with other work that indicates that ginger by itself and without LPS stimulated iNOS production in a dose dependent manner and in much lower doses (see American Journal of Chinese Medicine, Vol. 32, No. 5, pp 727-735, 2004). Nevertheless, experiments in vitro demonstrated that ZOR at low dosages in the presence of LPS did not appear to stimulate iNOS production. Other research has shown that ginger in vitro enhances iNOS activity, while ginger has been used in treatment of liver fibrosis (See Nutr Metab (Lond). 2011 Jun. 20; 8:40. doi: 10.1186/1743-7075-8-40. PubMed PMID: 21689445; PubMed Central PMCID: PMC3199745).

L-arginine, the amino acid that provides the nitrogen moiety for NO during its synthesis, has by itself or in combination with PDE5 inhibitors also shown to have some beneficial effects in minimizing fibrosis both in vivo and in vitro. In addition, the natural products Muira Puama and Paullinia cupana have been reported to enhance erectile function. Paullinia cupana exhibits in vitro protective effects against cytotoxicity and oxidative stress in NIH-3T3 embryonic fibroblasts cells induced by SNP exposure, suggesting that Paullinia cupana has an in vitro bioactive action on NO modulation. Nevertheless, prior to aforementioned International Publication WO 2012/067745 A1, insufficient research has been done on ginger, Muira Puama, Paullinia cupana and amino acids to enable reliable medical use of such compounds, alone or in combination with other ingredients. The present inventors sought to better elucidate the bioactivity of these substances, while also dealing with the complexity of solving complex and unpredictable scientific problems associated with ED and fibrotic diseases. These problems include finding compositions and methods for treatment and inhibition of ED and fibrotic diseases that are practical for long term routine administration, avoiding side effects of existing treatments, enabling treatment of and prevention of ED in patients that cannot utilize prior art ED treatments, and/or providing new practical and cost effective compositions to prevent as well as treat ED and fibrotic diseases.

### SUMMARY OF THE INVENTION

The present invention is directed to methods and compositions for extending the duration of efficacy of PDE-I compounds for treating ED in patients and/or extending duration of patient use of PDE-I compounds for treating ED. Compositions comprising ginger and L-Citrulline are administed in combination with PDE-I compound administration to extend the duration of efficacy of PDE-I compounds in treating ED. A preferred composition comprises ginger (“G”), Muira Puama (“M”), Paullinia cupana (“P”), and L-citrulline (and/or L-arginine) (“C”). L-citrulline is a preferred compound for producing L-arginine in vivo and can be used instead of or in addition to L-arginine. GC and GCMP compositions of the present invention are useful in treating ED, ARED, and fibrotic diseases, and can be used in place of the compositions taught in International Publication No. WO 2012/067745 A1. Further research discussed in the Figures and otherwise herein further substantiates the efficacy of the inventions disclosed in International Publication No. WO 2012/067745 A1, while also disclosing particularly effective compositions and methods for treating ED, ARED, and fibrotic diseases.

In an embodiment, a formulation comprising ginger, L-Citrulline, Muira Puama and Paullinia cupana has been found to be particularly effective in at least slowing, if not halting or reversing, the progression of cavernosal veno-occlusive dysfunction, smooth muscle cell (SMC) loss, corporal smooth muscle cell (CMC) loss, and corporal fibrosis. Compositions of the present invention, comprising ginger, L-citrulline and/or L-arginine, Muira Puama and Paullinia cupana (“GCMP”), can be used for the treatment of ED and ARED. GCMP compositions containing varying amounts of G, C, M and P may also be referred to herein as “COMP-4,” wherein “C” refers to L-citrulline. L-arginine, or compounds that produce L-arginine in vivo (unless otherwise indicated).

It was also surprisingly discovered that the GCMP compositions of the present invention can be combined with a PDE5 inhibitor to provide enhanced treatment of ED and ARED by PDE5-I compounds. In particular, combination therapy with a PDE5-I and a GCMP composition of the present invention provides improved results over treatment with PDE5-I alone. Thus use of GC and GCMP compositions of the present invention can be used to extend the duration of efficacy of PDE-I compounds in treating ED, as well as improve patient compliance with use of PDE-I compounds as recommended by their physicians. The GCMP compositions of the present invention were also surprisingly found to have improved histological benefits over PDE5-I, such as improved slowing or reversal of SMC loss (these and other benefits are described and illustrated below in the Experimental Section).

Thus, use of the compositions and methods of the present inventions can be effective in retarding and/or reversing the ongoing corporal SMC loss and fibrosis, and the subsequent CVOD or venous leakage, associated with aging. The present inventions and corresponding surprising research discoveries improve upon and reinforce earlier research findings that surprisingly discovered that ED and/or certain fibrotic diseases can be arrested, inhibited, or even reversed by use of compositions comprising ginger and L-citrulline (and/or L-arginine), as well as such formulations further comprising at least one of the group consisting of Muira Puama and Paullinia cupana. The present inventions include improved methods and compositions to treat and/or prevent ED. The compositions of the present inventions are particularly useful in the treatment of ARED. The new compositions and methods of the present invention use dosages of ginger small enough to be useful and practicable in routine, even daily, administration over an extended period of time for treating and inhibiting ED.

An exemplary preferred daily dosage GCMP composition of the present invention (for a 70 Kg man) comprises about 500 mg Muira Puama, 500 mg Paullinia cupana, 500 mg ginger (e.g., ginger rhizome) and about 1500 mg/day L-Citrulline. Note that “COMP4” used subsequently herein comprises a particular composition of GCMP.

In another embodiment, a PDE5 inhibitor (PDE5-I) composition is administered concomitantly with a GCMP composition for treating ED or ARED. An exemplary PDE5-I compound is tadalafil.

Treatment for ED includes repeated administration of the compositions of the present inventions to a patient over a period of time sufficient to obtain the desired result. Treatment with compositions of the present invention may be for prophylactic purposes, for slowing or halting progression of symptoms, or for temporary or longer-term reversal of symptoms. Certain preferred compositions of the present inventions do not contain compounds that require a prescription and/or avoid side effects of current “first line” treatments for ED. Other embodiments may require the use of compounds that can be obtained by prescription only. Below are preferred exemplary embodiments:

A composition, “A.” for treating erectile dysfunction, wherein a pharmaceutically effective amount of said composition administered periodically for a period of time sufficient for treatment comprises about 100 mg to about 2 grams ginger, about 300 mg to about 10 grams of a compound that produces L-arginine in vivo, about 100 mg to about 5 g Muira Puama, and about 100 mg to about 5 g Paullinia cupana.

Alternatively, in composition “B,” the compound in composition A that produces L-arginine in vivo is L-citrulline.

In an embodiment, a dose of composition B, referred to as composition “C,” administered about daily for a sufficient amount of time until reaching a desired level of treatment of erectile dysfunction, comprises about 500 mg ginger, about 1500 mg L-citrulline, about 500 mg Muira Puama and about 500 mg Paullinia cupana.

Compositions of the present invention, such as compositions A, B and C, can halt, slow or reverse the deterioration of corpora cavernosa smooth muscle cells in patients suffering from ED. The present inventions are particularly useful in treating age related erectile dysfunction.

A pharmaceutically effective amount of the compositions of the present invention, such as compositions A, B, and C, can be used as prophylaxis against erectile dysfunction, smooth muscle cell deterioration, and corporal smooth muscle cell deterioration.

A pharmaceutically effective amount of the compositions of the present invention, such as compositions A, B, and C, can be used for treating or inhibiting fibrotic diseases.

A pharmaceutically effective amount of the compositions of the present invention, such as compositions A, B, and C, can be used for treating or inhibiting arteriosclerosis or fibrosis of vascular smooth muscle.

A pharmaceutically effective amount of the compositions of the present invention, such as compositions A, B, and C, can be used to extend the refractory period between erections in an individual, and increase and/or prolong tumescence.

A pharmaceutically effective amount of the compositions of the present invention, such as compositions A, B, and C, can be combined with, or concomitantly administered with, a phosphodiesterase type 5 inhibitor (PDE5-I) compound to enhance the benefits of PDE5-I treatment.

Treatments can range from short term self administered oral dosages taken periodically at least once a day for substantial periods of time, or much higher dosages may require the aid of a nurse if not a physician, particularly if with concomitant therapy with a PDE5 inhibitor.

More details of the present inventions follow. To simplify explanation, some Latin terms are not always presented in italics, and certain words that are normally capitalized may alternatively be presented in lower case.

### DESCRIPTION OF THE FIGURES

FIG. 1 demonstrates prevention of cavernosal veno-occlusive disorder (CVOD) in middle-aged Fischer 344 rats, determined by dynamic infusion cavernosometry, by oral treatment with tadalafil (TAD), compositions of the present invention, in this example COMP4 (an exemplary Ginger, L-citrulline, Muira Puama, and Paullinia cupana, “GCMP,” formulation of the present invention), and a combination of COMP4 with TAD. The top bar graph shows intracavernosal pressure after papaverine administration (ICPAP) to induce an erection, while the bottom bar graph shows the drop rate in ICP. 5 MO refers to five month-old subjects, and 12 MO refers to twelve month-old subjects, at the time cavernosometry and euthanasia. COMP4 refers to animals treated with COMP4. TAD refers middle-aged animals treated with tadalafil, COMP4+TAD refers to middle-aged animals treated with a combination of composition 4 and tadalafil, at the time of cavernosometry and euthanasia. With respect to the following characters above the bars in the charts: ** is for p<0.01, * is for p<0.05 with respect to 12 MO, and ### is for p<0.005 with respect to 5-MO.

FIG. 2 illustrates partial normalization by TAD, and complete normalization by compositions of the present invention, in this example COMP4 as well as by a the combination COMP4+TAD, of the reduced smooth muscle/collagen ratio and increased collagen content in the middle aged rat corpora cavernosa. The pictures (40×, Bar=50 μm) are representative of penile corpora cavernosa tissue sections stained with Masson trichrome for the rat groups in FIG. 1. The bar graph below the pictures results from quantitative image analysis of the SMC/collagen ratio. With respect to the following characters above the bars in the charts: * is for p<0.05, ** is for p<0.01, and *** is for p<0.005 with respect to 12 MO.

FIG. 3 illustrates the effect of TAD, compositions of the present invention, in this example COMP4 and COMP4+TAD, on the preservation of smooth muscle content in the middle-aged rat corpora cavernosa. The pictures (200×, Bar=50 μm) are representative of penile corpora cavernosa sections adjacent to those presented in FIG. 2 that are immunostained for desmin as a smooth muscle cell marker. The bar graph below the pictures results from quantitative image analysis. With respect to the following characters above the bars in the chart: * is for p<0.05, and ** is for p<0.01, with respect to 12 MO.

FIG. 4 illustrates that iNOS expression in the middle-aged rat corpora cavernosa is up regulated by COMP4 as well as by COMP4-TAD. The pictures (200×, Bar=50 μm) are representative of penile corpora cavernosa sections adjacent to those presented in previous figures that were subjected to immunodetection for iNOS. The bar graph below the pictures results from quantitative left image analysis.

FIG. 5 illustrates that GCMP compositions of the present invention, in this example COMP4, reduce oxidative stress in whole blood of middle-aged rats. Oxidative stress was determined by measuring GSSG and GSH in whole blood after the addition or not of M2VP respectively. The GSH/GSSG ratio was calculated as: (GSH-2*GSSG)/GSSG. The character * is for p<0.05 with respect to 12 MO.

### FURTHER DETAILS OF THE INVENTION

Further research is set forth below, including methodology and conclusions. In particular, it was surprisingly discovered that oral administration of a composition comprising L-citrulline, ginger, muira puama and Paullinia cupana appeared effective in either retarding and/or reversing histological and functional characteristics of ARED in a manner similar to that seen experimentally with PDE5 inhibitors. In addition to treating ED, ARED, penile muscle degration, etc., the compositions of the present inventions can also be utilized in the treatment of cardiovascular disease. For example, compositions and methods of the present inventions can be used for treating cardiovascular disease manifesting in hypertension, as well as arteriosclerosis. Repeated dosages of compositions of the present inventions can be provided over extended periods of time until the desired effect is obtained. Repeated administration of the compositions of the present inventions to patients demonstrating ED symptoms can be made until desired ED treatment results are obtained, and may be continued thereafter as a prophylactic. The compositions can also be used as a prophylactic against eventual ED symptom manifestation in patients likely to demonstrate same.

Since it is believed that the fibrotic processes within the penis that are associated with ED may begin at any age, administration of the compositions of the present invention to a patient that has not noticed an increase in the refractory period, decrease in duration of erection, and/or insufficient tumescence of erection may also be referred to as halting or delaying the onset ED. However, since almost all men will ultimately develop some form of ED with age, and by age 40 a significant percentage of men experience noticeable symptoms of ED, so what may be referred to as prevention is likely a treatment that slows or stops further progression of the causes of ED. Preferably, as part of a daily vitamin or other nutrition routine, a pharmaceutically effective amount of compositions of the present invention are taken regularly, if not daily, by males between the age of 20 and 40 (if not sooner), while males over 40 should regularly be treated.

Certain preferred compositions of the present inventions comprise compounds found in foods or extracted from foods, and thus may be referred to as “nutraceuticals.” While nutraceutical compositions have traditionally been found in a medicinal format, such as capsules or tablets, an increasing number of foods have been fortified with nutraceuticals. Analogs and/or homologs of ginger constituents that have activity in promoting iNOS sufficient to ameliorate, stop or reverse fibrotic events associated with ED may also be used in combination with L-arginine and/or L-citrulline. The present inventions can therefore be administered in a wide variety of ways and forms matching the lifestyle and dietary preferences of the users.

In an embodiment, testing for ED is performed with a questionnaire called the IIEF (International index of erectile function or similar terminology). Patients on any treatment regimen for ED may be followed with this IIEF scoring system and this can be performed on a periodic or annual basis to monitor progression of the ED. See “The international index of erectile function (IIEF): a multidimensional scale for assessment of erectile dysfunction,” Rosen R C, Riley A, Wagner G, Osterloh I H, Kirkpatrick J, Mishra A. Urology. 1997 June; 49(6):822-30. Physicians and scientists involved in the study and/or treatment of male sexual dysfunction have found the IIEF to be a reliable scientific metric.

### EXPERIMENTAL

Based on the beneficial effects of the compositions disclosed in the aforementioned International Publication WO 2101/067745 A1, experiments were designed and conducted to determine whether, in middle aged rats, the daily oral administration of a combination of ginger, paullinia cupana, muira puama and L-citrulline (as a substitute for L-arginine) can be effective in retarding and possibly reversing the ongoing corporal SMC loss and fibrosis, and the subsequent corporal veno-occlusive dysfunction CVOD associated with aging. The efficacy of this GCMP combination was compared to the known anti-oxidant and anti-fibrotic effects with chronic PDE5 treatment.

Materials and Methods

Animals and Treatments:

Middle aged 10 month-old Fisher 344 rats (Harlan Sprague-Dawley, San Diego, Calif.) were divided in 4 groups and treated daily for two months as follows:
a) Control: 12 month-old at the time of death receiving by retrolingual administration vehicle only composed of 10% dimethyl sulfoxide, peanut butter and water (12 month);
b) Composition 4 (COMP 4): a combination of Muira Puama (45 mg/Kg B.W), Paullinia cupana (45 mg/Kg B.W), ginger (45 mg/Kg B.W) and L-Citrulline (133 mg/Kg B.W) dissolved in vehicle;
c) Tadalafil (TAD): 2.5 mg/Kg B.W tadalafil dissolved in vehicle;
d) Composition 4+Tadalafil (COMP4+TAD): A combination of b and c at the same doses.

DOSAGES: Doses were corrected for body surface to be equivalent to a human dose of 500 mg/day ginger rhizome, 1500 mg/day of L-Citrulline, 500 mg/day Paullinia cupana, and 500 mg/day Muira Puama in a 70 Kg man. These doses are below the toxicity level for each drug. Tadalafil dose was also corrected for body surface and is equivalent to 22 mg/day in a 70 Kg man. This dose is slightly higher than the maximum recommended dose in humans and is one-half of what we have used in previous studies.

Testing:

Dynamic Infusion Cavernosometry was performed following techniques well described in the literature, so the technique is only briefly described. Under deep anesthesia the penis was exposed, a cannula inserted into the corpora cavernosa and the basal intracavernosal pressure (ICP) was recorded. Papaverine (5 mg/Kg) was administered via the cannula. The ICP during tumescence was recorded as ICP after papaverine (ICPAP). Saline was then infused through another cannula, increasing infusion rate by 0.05 ml/min every 10 seconds, until the ICP at 80 mmHg was reached the maintenance rate at 80 mmHg. The “drop rate” was then determined by recording the fall in ICP from 80 mmHg within one minute after the infusion was stopped.

Histochemistry and Immunohistochemistry. After cavernosometry, animals were euthanized and the skin-denuded middle part of penile shafts was fixed overnight in 4% buffered p-formaldehyde, and stored in alcohol 70% at 4 C until processed for paraffin embedded tissue sections. Five micrometer anatomical matched sections were used for:
a) Masson trichrome staining for collagen (blue) and SM (red);
b) immunodetection with: polyclonal antibody against desmin, a marker of smooth muscle cell content.
c) polyclonal antibody against iNOS (Calbiochem, La Jolla, Calif.). The specificity of the antibodies was validated by western blot.

Sections were then incubated with biotinylated anti-Rabbit IgG followed by ABC complex (Vector labs, Temecula, Calif.) and 3,3′diaminobenzidine (Sigma). Sections were counter-stained with haematoxylin. Negative controls of the immunohistochemical detections were done by replacing the first antibody with IgG isotype.

Quantitative Image Analysis was performed by computerized densitometry using ImagePro 7.1 software (Media Cybernetics, Silver Spring, Md.), coupled to an Olympus BHS microscope equipped with a Retiga digital camera. For Masson staining, 40× magnification pictures of the penis comprising half of the corpora cavernosa were analyzed for smooth muscle (stained in red) and collagen areas (stained in blue) excluding the sinusoidal spaces, and expressed as the smooth muscle/collagen ratio. For desmin and iNOS staining, 200× magnification pictures of the corpora cavernosa were analyzed in a computerized grid and expressed as a percentage of positive area versus total area of the corpora cavernosa. In all cases, four fields at 40×, or eight fields at 200×, were analyzed per tissue section, with at least 4 matched sections per animal and 6 animals per group.

Estimation of Gluthatione reduced (GSH) and Gluthatione oxidized (GSSG). The GSH/GSSG ratio is inversely proportional to oxidative stress levels and monitors the effectiveness of antioxidant intervention strategies. For the measurement of GSH/GSSG ratio, blood was collected with or without 1-methyl-2 vinylpyridinium trifluoromethane sulphonate (M2VP) scavenger of reduced glutathione, described in the commercial kit protocol (‘Bioxytech GSH/GSSG-412 kit’ from Oxis Health Products, Portland, Or). The omission or addition of M2VP allows the measurement of GSH and GSSG, respectively. Spectrophotometric detection was recorded at 412 nm for 3 min after the addition of 3.8 μmol NADPH.

Statistical Analysis. Values are expressed as Mean±SEM. The normality distribution of the data was established using the Wilk-Shapiro test. Multiple comparisons were analyzed by a single factor analysis of variance (ANOVA), followed by post-hoc comparisons with the Student-Neuman-Keuls test, according to the GraphPad Prism V 5.1. Differences were considered significant at P<0.05. Data for five month-old animals was obtained from studies with five month-old non-treated animals.

Data and Graphical Analysis.

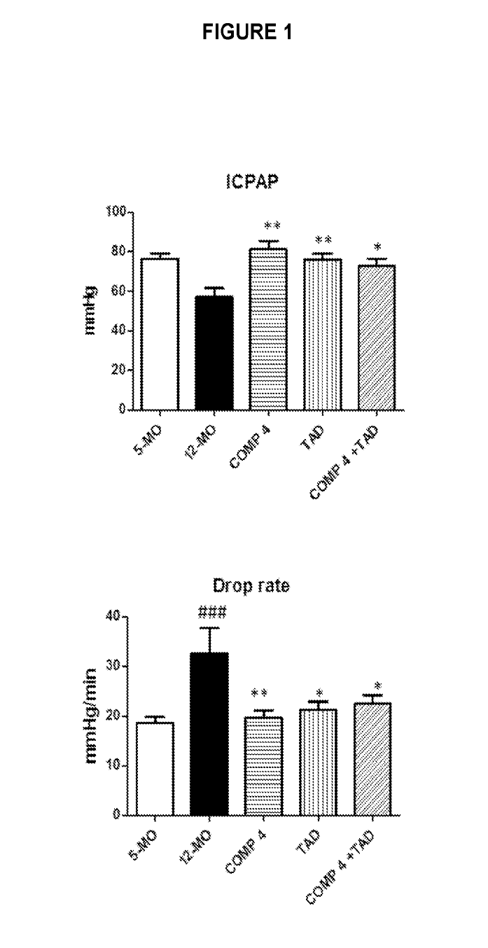

With reference to FIG. 1, top and bottom bar chart graphs show that control 12 month-old rats that received only vehicle had a considerable decrease (28%) in ICP (intracavernosal pressure) after intracavernosal papaverine administration (top graph), and a concomitant 1.7-fold increase in the drop rate (bottom) when compared to previously published data in young 5 mo old (young) control animals. This indicates the presence of ED (as measured by the ICPAP) and a moderate venous leak or CVOD (as measured by the drop rate) in the 12 month-old rats. The treatment of these 10 mo old animals for 2 months with daily PDE5 inhibitor, tadalafil, increased the papaverine response (top graph) and decreased the drop rate (bottom graph) when compared to the 12 mo control animals. This is in agreement with previous results in aged animals treated daily via drinking water with another PDE5 inhibitor, sildenafil. Daily administration of COMP-4 for two months increased the papaverine-induced erection (top) and reduced the drop rate (bottom) to values not significantly different from the ones treated with daily tadalafil or the young 5 month-old non-treated animals. The combination of COMP-4 plus TAD was similar to the ICPAP response for either COMP4 alone or TAD alone.

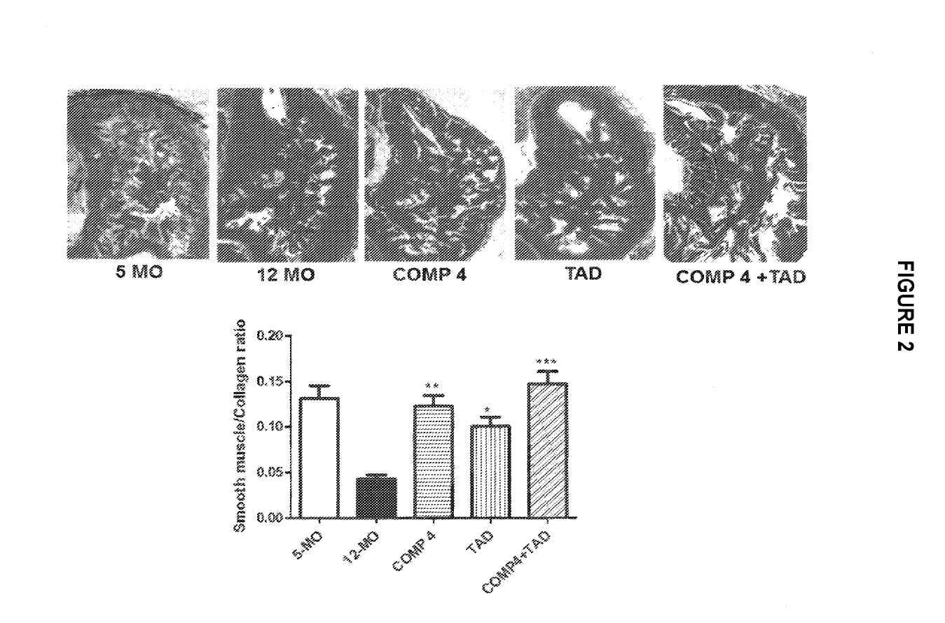

Referring to FIG. 2, the presence of venous leakage or CVOD as determined by the drop rate observed in the 12 month-old control animals was also accompanied by a 70% decrease in the smooth muscle (SMC)/collagen ratio as determined by Masson trichrome staining. Further, in agreement with previous published results with daily and continuous sildenafil in drinking water, daily oral treatment with the PDE5 inhibitor TAD improved the SMC/collagen ratio by 60% when compared to the 12 month-old control animals, although the ratio still remained lower than that seen in the 5 month-old control. However, surprisingly, daily treatment with COMP-4 alone restored the SMC/collagen ratio to levels similar to those of the young 5 month controls while the combination of COMP4 with TAD further improved the levels above the 5 month controls.

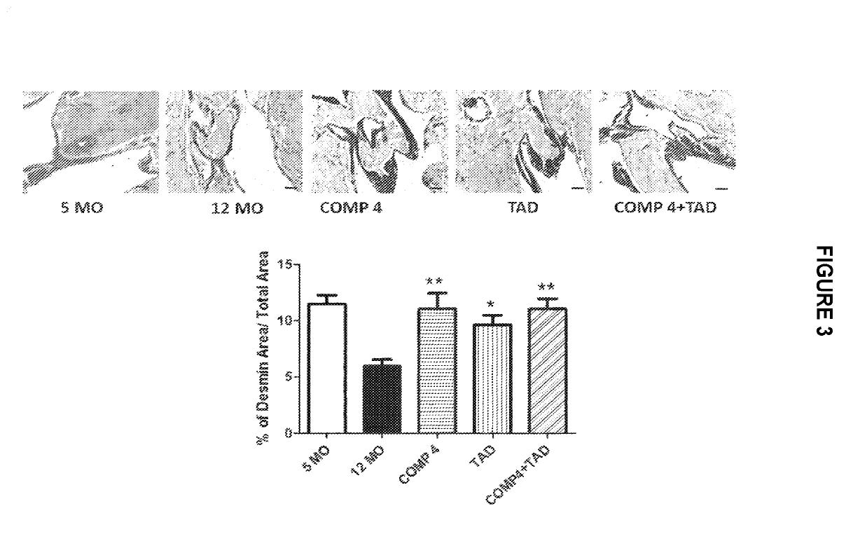

Referring to FIG. 3, to determine the effect of treatment for two months with COMP4 with or without TAD had solely on the smooth muscle content of the corpora of the 10 month-old animals, a marker of smooth muscle, desmin, was evaluated by immunohistochemistry in adjacent sections to those employed to determine the aforementioned SMC/collagen ratio. FIG. 3 shows as expected a significant 52% reduction of desmin expression at 12 months when compared to the 5 month animals. Treatment for 2 months with either COMP4 or COMP4+TAD resulted in a marked increase in desmin expression approaching levels seen in the young 5 month-old animals. As would be expected, there is a 38% increase in the desmin expression in the TAD treated group when compared to the 12 month-old non-treated controls, but this level does not quite reach those levels seen in young 5 month-old animals. Furthermore, COMP-4 and COMP4+TAD groups produced a more pronounced effect with an increase in desmin expression by 54% with respect to the 12 month-old-group reaching the same level of expression as 5 month-old control group.

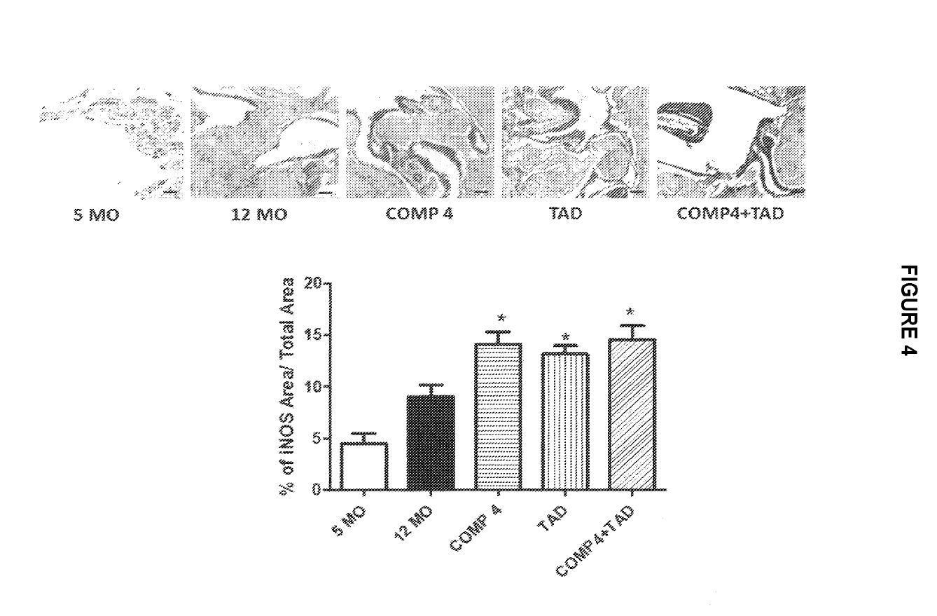

FIG. 4 shows the results of experiments to elucidate whether the improvement in penile dynamics and the reduction of fibrosis seen in the aforementioned treated animals are mediated by an increase in iNOS expression, which was assessed by immunohistochemistry. As expected with aging, there was a significant increase in iNOS expression in the 12 month-old control animals with respect to 5 month-old controls. With daily tadalafil for 2 months, there was a non-significant but slight increase in iNOS expression compared to the control 12 month-old animals. However, treatment with COMP4 produced a significant increase of 36% when compared to the 12 month-old controls. The combination of COMP4+TAD showed a similar significant increase in iNOS expression as the COMP4 group alone when compared to the 12 month-old controls.

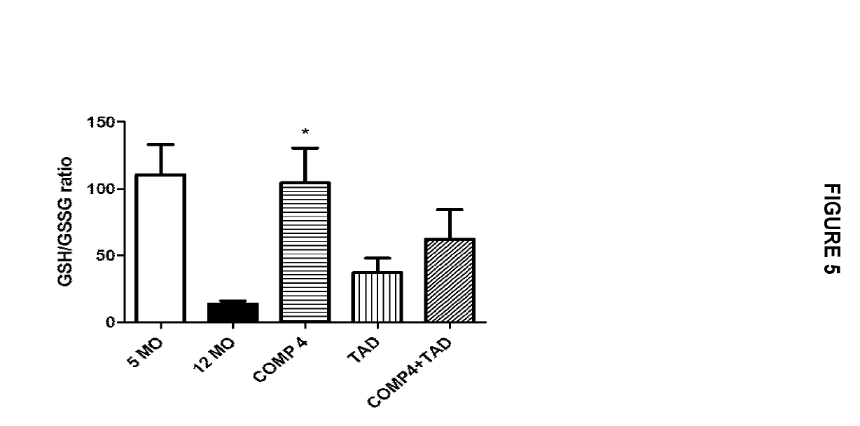

Referring to FIG. 5, to determine whether the surprising improvement of erectile function and reduction in corporal fibrosis observed by the treatment with COMP4 for two months can be attributed to a reduction in oxidative stress, which is one of the presumptive mechanisms by which NO from iNOS exerts its anti-fibrotic effect, the GSH/GSSG ratio was determined in the whole blood of these animals. The GSH/GSSG ratio is a method of measuring oxidative stress.

FIG. 5 shows the 12 month-old non-treated control animals exhibited levels indicative of considerable oxidative stress levels (a very low ratio) when compared to young 5 month-old animals (a high ratio). Two months of daily treatment with COMP4 effectively increased the GSH/GSSG ratio to levels (less oxidative stress) similar to those found in 5 month-old animals. The TAD and COMP4+TAD animals also showed increases in the ratio but did not achieve the levels seen in the young 5 month-old or the 12 month-old COMP4 treated animals.

The foregoing results indicate that in the laboratory rat a decline in the rat's erectile function as measured by both by the ICPAP and the drop rate is evident by 10-12 months of age. This decline is accompanied histologically by a decrease in the corporal smooth muscle content, its replacement by collagen and a resultant increase in corporal fibrosis. Daily treatment of these animals for two months with COMP-4 reverses this process; CVOD or venous leakage as measured by the drop rate is improved, smooth muscle content is increased, and corporal fibrosis is reduced. In addition, COMP4 reduced oxidative stress as measured by the GSG/GSSG levels in the blood.

Without being limited to any particular theory of operation, it is believed that aging related ED occurs in an environment of high oxidative stress and may be due to a genetically predetermined apoptosis of the corporal smooth muscle with replacement of the apoptotic cells by collagen resulting in an increase in corporal fibrosis. The SMC responds to high oxidative stress and the apoptotic process by inducing iNOS, which theoretically produces high levels of intracellular NO that can act as an anti-oxidative and an anti-fibrotic molecule. In a previously published study, a markedly aged rat of about 21 months of age treated daily with the PDE5 inhibitor, sildenafil, which presumably acts on the NO-cGMP pathway to enhance the effect of NO, showed halting of the aging related corporal SMC apoptosis and apparent reduction of the formation of fibrosis within the corpora. In addition, in the cavernosal nerve resection rat model in which the animals were about 5 months old, it has been shown experimentally that continuous daily treatment with PDE5 inhibitors can retard the apoptosis of the corporal SMC and minimize the development of corporal fibrosis normally seen when the cavernosal nerve is iatrogenically damaged. In addition, in Peyronie's disease which is another penile fibrotic condition albeit in the tunica albuginea rather than in the corporal tissue, gene therapy with an iNOS cDNA has been shown to reverse the fibrosis that occurs in this experimentally induced Peyronie's disease rat model.

Thus, the rat model and the present experiments are valid predictors of human efficacy of the present inventions. COMP4 reversed and/or halted histological changes in the penis, thus demonstrating a potent treatment for treating corporal fibrosis as well as other fibrotic diseases. When COMP4 was given together with tadalafil, this combination had a similar effect as COMP4 alone on the ICPAP, drop rate and desmin staining. The SMC/collagen ratio of the COMP4+TAD animals was slightly better than TAD alone, so where a PDE5-I compound can be tolerated, combination of COMP4+TAD can produce better outcomes in the amelioration of corporal fibrosis. The data supports prior animal studies with the PDE5-I compound sildenafil, wherein the negative histological changes associated with ARED were offset.

Further, treatment with either COMP4, TAD by itself or the combination of TAD+COMP4 increased iNOS expression. Since iNOS can lead to NO output that acts as an antifibrotic defense mechanism by inhibiting collagen synthesis and myofibroblast differentiation, the present inventions are further demonstrated as being useful in treating fibrotic disease. This is supported by research on Peyronie's disease, where treatment with an iNOS cDNA gene ameliorated the fibrosis, whereas the administration of an iNOS inhibitor (L-N-[1-iminoethyl]-lysine,acetate) enhanced the fibrosis. As such, for example, COMP4 is useful in either reversing or delaying the onset or progression of ARED.

Thus, it has been established that compositions of the present inventions improve erectile function in aging by combating those aging related physiological processes that impact the normal functioning of the corporal tissue. The forgoing experiments using a combination of COMP-4 with the PDE5 inhibitor tadalafil, shows that the use of natural interventions (e.g., a “nutraceutical” of the present invention) can have similar effects and outcomes, if not better, as PDE5 inhibitors at a potentially much reduced cost.

While details of certain embodiments of the present inventions are described, they are provided as illustrative examples so as to enable those of ordinary skill in the art to practice the inventions. The details provided are not meant to limit the scope of the present inventions, but to be exemplary. Where certain elements of the present inventions can be partially or fully implemented using known constituents, only those portions of such known constituents that are necessary for an understanding and making of the present invention are described, and detailed descriptions of other constituents or formulating processes are omitted as being to simplify explanation of the invention. Further, the present invention encompasses present and future known equivalents to the compositions and methods referred to herein. The inventions are capable of other embodiments and of being practiced and carried out in various ways, and as such, those skilled in the art will appreciate that the conception upon which this disclosure is based may readily be utilized as a basis for the designing of other methods and compositions for carrying out the several purposes of the present inventions.

Commercial formulations of the present invention can be made using technologies and processes conventionally used in producing pharmaceuticals, vitamins, and nutraceuticals, and such are incorporated herein.

Additional References Listing: * Garban H, Vernet D, Freedman A, Rajfer J, Gonzalez-Cadavid N F. Effect of aging on nitric oxide-mediated penile erection in the rat. Am J Physiol 1995; 268:H467-H475. * Bakircioglu M E, Sievert K D, Nunes L, Lau A, Lin C S, Lue T F. Decreased trabecular smooth muscle and caveolin-1 expression in the penile tissue of aged rats. J Urol 2001; 166:734-738. * Ferrini M, Magee T R, Vernet D, Rajfer J, Gonzalez-Cadavid N F. Aging related expression of inducible nitric oxide synthase (iNOS) and markers of tissue damage in the rat penis. Biol Reprod 2001; 64:974-982. * Nehra A, Goldstein I, Pabby A, Nugent M, Huang Y H, de las Morenas A, Krane R J, Udelson D, Saenz de Tejada I, Moreland R B. Mechanisms of venous leakage: a prospective clinicopathological correlation of corporeal function and structure. J Urol. 1996 October; 156(4):1320-9. * Valente E G, Vernet D, Ferrini M G, Qian A, Rajfer J, Gonzalez-Cadavid N F. L-arginine and phosphodiesterase (PDE) inhibitors counteract fibrosis in the Peyronie's fibrotic plaque and related fibroblast cultures. Nitric Oxide. 2003 December; 9(4):229-44. PubMed PMID: 14996430. * Davila H H, Magee T R, Rajfer J, Gonzalez-Cadavid N F. Gene therapy with the inducible nitric oxide synthase (iNOS) cDNA regresses the fibrotic plaque in an animal model of Peyronie's disease. Biol Reprod 2004; 71:1568-1577. * Ferrini M G, Kovanecz I, Nolazco G, Rajfer J, Gonzalez-Cadavid N F. Effects of long-term vardenafil treatment on the development of fibrotic plaques in a rat model of Peyronie's disease. BJU Int. 2006 March; 97(3):625-33. PubMed PMID:16469038. * Ferrini M G, Kovanecz I, Sanchez S, Vernet D, Davila H H, Rajfer J, Gonzalez-Cadavid N F. Long-term continuous treatment with sildenafil ameliorates aging-related erectile dysfunction and the underlying corporal fibrosis in the rat. Biol Reprod. 2007 May; 76(5):915-23. Epub 2007 Feb. 7. PubMed PMID: 17287493. * Ferrini M G, Davila H, Kovanecz I, Sanchez S, Gonzalez-Cadavid N F, Rajfer J. Long-term continuous treatment with vardenafil prevents fibrosis and preserves smooth muscle content in the rat corpora cavernosa after bilateral cavernosal nerve transection. Urology 2006; 68:429-435. * Ferrini M G, Kovanecz I, Sanchez S, Umeh C, Rajfer J, Gonzalez-Cadavid N F. Fibrosis and loss of smooth muscle in the corpora cavernosa precede corporal veno-occlusive dysfunction (CVOD) induced by experimental cavernosal nerve damage in the rat. J Sex Med. 2009 February; 6(2):415-28. doi: 10.1111/j.1743-6109.2008.01105.x. Epub 2008 Dec. 2. PubMed PMID: 19138364; PubMed Central PMCID: PMC2756287. * Sirad F, Hlaing S, Kovanecz I, Artaza J N, Garcia L A, Rajfer J, Ferrini M G. Sildenafil promotes smooth muscle preservation and ameliorates fibrosis through modulation of extracellular matrix and tissue growth factor gene expression after bilateral cavernosal nerve resection in the rat. J Sex Med. 201 April; 8(4):1048-60. doi: 10.1111/j.1743-6109.2010.02195.x. Epub 2011 Jan. 26. PubMed PMID: 21269401; PubMed Central PMCID: PMC3075874. * Kovanecz I, Rambhatla A, Ferrini M G, Vernet D, Sanchez S, Rajfer J, Gonzalez-Cadavid N. Chronic daily tadalafil prevents the corporal fibrosis and veno-occlusive dysfunction that occurs after cavernosal nerve resection. BJU Int. 2008 January; 101(2):203-10. Epub 2007 Sep. 20. * Bloch A, Thomson C A. Position of the American Diabetic Association: phytochemicals and functional foods. J Am Diet and Assoc. 1995; 95:406-493. * Imanishi N, Mantani N et al. Inducible activity of ginger rhizome on the mRNA expression of macrophage-inducible nitric oxide (NO) synthase and NO production in a macrophage cell line, RAW 264.7 cells. Am J of Chinese Medicine 32:727-735, 2004. * Liao H, Banbury L K and Leach D N: Elucidation of Dan Wan and its constituent herbs on antioxidant activity and inhibition of NO production eCAM advanced publication Jan. 9, 2007. doi: 10.1093/ecam/ne1091. * Motawi T K, Hamed M A, Shabana M H, Hashem R M, Aboul Naser A F. Zingiber officinale acts as a nutraceutical agent against liver fibrosis. Nutr Metab (Lond). 2011 Jun. 20; 8:40. doi: 10.1186/1743-7075-8-40. PubMed PMID: 21689445; PubMed Central PMCID: PMC3199745. * Valente E G, Vernet D, Ferrini M G, Qian A, Rajfer J, Gonzalez-Cadavid N F. L-arginine and phosphodiesterase (PDE) inhibitors counteract fibrosis in the Peyronie's fibrotic plaque and related fibroblast cultures. Nitric Oxide. 2003; 9(4):229-244. PubMed PMID: 14996430. * Al Qadi-Nassar B, Bichon-Laurent F, Portet K, Tramini P, Arnoux B, Michel A. Effects of L-arginine and phosphodiesterase-5 inhibitor, sildenafil, on inflammation and airway responsiveness of sensitized BP2 mice. Fundam Clin Pharmacol. 2007; 21(6):611-620. PubMed PMID: 18034662. * Yildirim A, Ersoy Y, Ercan F, Atukeren P, Gumustas K, Uslu U, Alican I. Phosphodiesterase-5 inhibition by sildenafil citrate in a rat model of bleomycin-induced lung fibrosis. Pulm Pharmacol Ther. 2010 June; 23(3):215-21. doi: * Rajfer J, Gore J L, Kaufman J, Gonzalez-Cadavid N. Case report: Avoidance of palpable corporal fibrosis due to priapism with upregulators of nitric oxide. J Sex Med. 2006 January; 3(1):173-176. PubMed PMID: 16409234. * Rowland D, Tai W. A review of plant-derived and herbal approaches to the treatment of sexual dysfunctions. J Sex Marital Ther. 2003 May-June; 29(3):185-205. Review. PubMed PMID: 12851124. * Bittencourt L S, Machado D C, Machado M M, Dos Santos G F, Algarve T D, Marinowic D R, Ribeiro E E, Soares F A, Barbisan F, Athayde M L, Cruz I B. The protective effects of guarana extract (Paullinia cupana) on fibroblast NIH-3T3 cells exposed to sodium nitroprusside. Food Chem Toxicol. 2013 March; 53:119-* Ferrini M G, Moon J, Rivera S, Rajfer J, Gonzalez-Cadavid N F. Amelioration of diabetes-indueced cavernosal fibrosis by antioxidant and anti-transforming growth factor-B1 therapies in inducible nitric oxide synthase-deficient mice. BJU Int 2012; 109:586-93. * Gonzalez-Cadavid N F, Rajfer J. The pleiotropic effects of inducible nitric oxide synthase (iNOS) on the physiology and pathology of penile erection. Curr Pharm Des. 2005, 11:4041-4046. * Kovanecz I, Rambhatla A, Ferrini M, Vernet D, Sanchez S, Rajfer J, Gonzalez-Cadavid N. Long-term continuous sildenafil treatment ameliorates corporal veno-occlusive dysfunction (CVOD) induced by cavernosal nerve resection in rats. Int J Impot Res. 2008 March-April; 20(2):202-12. Epub 2007 Sep. 20. PubMed PMID: 17882231. * Rambhatla A, Kovanecz I, Ferrini M, Gonzalez-Cadavid N F, Rajfer J. Rationale for phosphodiesterase 5 inhibitor use post-radical prostatectomy: experimental and clinical review. Int J Impot Res. 2008 January-February; 20(1):30-4. Epub 2007 Aug. 2. Review. PubMed PMID: 17673932. * Sani N F, Belani L K, Sin C P, Rahman S N, Das S, Chi T Z, Makpol S, Yusof Y A Effect of the combination of gelam honey and ginger on oxidative stress andmetabolic profile in streptozotocin-induced diabetic Sprague-Dawley rats. Biomed Res Int. 2014; 2014:16069 * Schwedhelm E, Maas R, Freese R et al. Pharmacokinetic and pharmacodynamic porperties of oral L_citrulline and L-arginine: impact on nitric oxide metabolism. Brit J of Clin Pham 65:51-59, 2007. * Antunes E, Gordo W M, de Oliveira J F, Teixeira C E, Hyslop S, De Nucci G. The relaxation of isolated rabbit corpus cavernosum by the herbal medicine Catuama and its constituents. Phytother Res. 2001(5):416-421. * Otunola G A, Oloyede O B, Oladiji A T, Afolayan A J. Selected spices and their combination modulate hypercholesterolemia-induced oxidative stress in experimental rats. Biol Res. 2014; 47(1):5. * Vernet D, Ferrini M G, Valente E G, Magee T R, Bou-Gharios G, Rajfer J, Gonzalez-Cadavid N F. Effect of nitric oxide on the differentiation of fibroblasts into myofibroblasts in the Peyronie's fibrotic plaque and in its rat model. Nitric Oxide. 2002; 7(4):262-276. * Ferrini M G, Vernet D, Magee T R, Shahed A, Qian A, Rajfer J, Gonzalez-Cadavid N F. Antifibrotic role of inducible nitric oxide synthase. Nitric Oxide. 2002; 6(3):283-94. PubMed PMID: 12009846.

1. A composition for treating erectile dysfunction, wherein a pharmaceutically effective amount of said composition comprises about 100 mg to about 2 grams ginger, about 300 mg to about 10 grams of a compound that produces L-arginine in vivo, about 100 mg to about 5 g Muira Puama, and about 100 mg to about 5 g Paullinia cupana.

2. The composition of claim 1, wherein said composition that produces L-arginine in vivo is L-citrulline.

3. The composition of claim 1, wherein a dose of same administered about daily for a sufficient amount of time to reach a desired level of treatment of erectile dysfunction comprises about 500 mg ginger, about 1500 mg L-citrulline, about 500 mg Muira Puama and about 500 mg Paullinia cupana.

4. The composition of claim 1, wherein said composition treats erectile dysfunction by halting, slowing or reversing the deterioration of smooth muscle cells of the corpora cavernosa.

5. The composition of claim 4, wherein a pharmaceutically effective amount of said composition is used as a prophylaxis against erectile dysfunction.

6. The composition of claim 4, where a pharmaceutically effective amount of said composition is used for treating or inhibiting fibrosis.

7. The composition of claim 4, where a pharmaceutically effective amount of said composition is used for treating or inhibiting arteriosclerosis.

8. The composition of claim 1, wherein said composition further comprises a phosphodiesterase type five inhibitor compound.

9. The composition of claim 1, wherein said composition can be administered concomitantly with a phosphodiesterase inhibitor compound to enhance the long term benefits of phosphodiesterase inhibitor compound treatment.

10. A method of treatment comprising the use of a composition to extend the duration of efficacy of a phosphodiesterase inhibitor compound to treat erectile dysfunction in a patient, the composition comprising a
pharmaceutically effective amount of a composition comprising about 100 mg to about 2 grams ginger, and about 300 mg to about 10 grams of a compound that produces L-arginine in vivo, wherein said use comprises the steps of administering said composition over a period of time sufficient to extend the duration of efficacy of a phosphodiesterase inhibitor in treating symptoms of erectile dysfunction in the patient, said composition being administered before, during, or following phosphodiesterase inhibitor administration.

11. The method of claim 10, wherein said compound that produces L-arginine in vivo comprises at least one compound selected from the group consisting of L-arginine and L-citrulline.

12. The method of claim 10, wherein said composition further comprises at least one of the group consisting of Muira Puama and Paulinia cupana

13. The method of claim 11, wherein said composition further comprises at least on of the group consisting of about 100 mg to about 5 g Muira Puama, and about 100 mg to about 5 g Paullinia cupana.
